(19) United States
(12) Patent Application Publication
(10) Pub. No.: US 2006/0254595 A1
(43) Pub. Date: Nov. 16, 2006

(54) Endotracheal positioning device

(76) Inventor: James Lee Rea, Ventura, CA (US)

Correspondence Address: Richard J. Grundstrom, P. O. Box 26, Holts Summit, MO, 65043, US

(21) Appl. No.: 11/129,593

(22) Filed: May 13, 2005

(51) Int. Cl. A62B 9/06; A61M 16/00

(52) U.S. Cl. 128/207.14; 128/207.15; 128/207.16; 128/200.26

(57) ABSTRACT

An endotracheal positioning device having an elongated triangular shape with a superior surface that projects towards the junction of the vocal cords, a posterior surface that projects towards the endotracheal tube or the posterior-interior surface of the cricoid cartilage, and generally straight right and left lateral surfaces that project anterior lateral on either side toward the vocal cords. The lateral surface are of appropriate dimensions for attachment of laryngeal surface electrodes. The device has a center opening or channel that may wrap around, slide over, or may otherwise be attached to an endotracheal tube or to a high pressure ventilatory apparatus of any type. The device has an attachment or docking recess on its proximal aspect for an actuating rod or a fiber optic laryngoscope. This allows continuous viewing of the larynx for positioning, insertion, and continuous monitoring of the vocal cords during surgical procedures.

### BACKGROUND OF THE INVENTION

The present invention relates to an endotracheal positioning device and more specifically a positioning device that provides optimal positioning of a surface electrode against the vocal cord muscles for laryngeal surgical electromyographic monitoring and visual monitoring. The device is a carrier for laryngeal surface electrode(s) with provisions for attaching a fiber optic laryngoscope.

There are several electrode systems that have been advocated for laryngeal evoked electromyographic (EMG) for recurrent and external nerve preservation. They are classified as invasive (needle) or noninvasive (surface) electrodes.

The advantages of needle electrodes are more precise localization of the EMG sample to the needle tip, and possible enhanced security of the electrode-muscle connection. Simple needle electrodes, and fine-wire or hook-wire electrodes have been used in the glottis. They are placed by direct laryngoscopy and may be placed into the vocalis muscle (vocal cords) or the transverse arytenoids muscles. This method requires considerable skill and carries the constant risk of abscess or hematoma of the larynx. In Germany surgeons have done thousands of cases using needle electrodes placed blindly through the cricothyroideus membrane into the vocalis muscle. This method has fewer drawbacks than the direct laryngoscope approach but still requires considerable practice and skill in electrode placement. Again, since the procedure is nonvisual the possibility of hematoma, abscess, misplacement, or needle breakage is present.

There are two commercial surface electrodes for laryngeal monitoring, the Medtronic integrated endotracheal tube electrode, with two pairs of bare wires facing each vocal cord, and the Neurovision Medical Products attachment endotracheal tube electrode (U.S. Pat. No. 5,178,145 issued to Rea) with a single electrode plate facing each vocal cord. There is a learning curve for placement of endotracheal tube electrodes, with misplacement causing annoying delay of the procedure in order to correct the problem, or suspension of the use of EEMG.

An additional monitoring modality is to use a surface EMG electrode in the postcricoid location. In this case the electrode is attached to a soft paddle and placed by laryngoscopy behind the larynx, the posterior cricoarytenoideus muscles resting on top of the electrodes. This monitors the largest muscles of the larynx, and the only pure abductors. Similar to the case of the ET tube electrode, the postcricoid placement requires a learning curve to properly use the device. Certain conditions may indicate the need for “multiple-loci” monitoring where an EMG unit is used with one channel on the endotracheal tube and one on a postcricoid paddle.

Both invasive and noninvasive systems have disadvantages. The endotracheal tube borne electrode can be difficult to position and maintain for monitoring purposes, leading to a substantial percentage of uses with failure to monitor. Modifications of the design of the electrode, primarily in increasing its size, and the protocol for positioning have improved the percentages but have not reduced the failure rate to zero. Needle electrodes may be intrinsically more reliable for monitoring purposes but the blind puncture of the vocal cord from above inevitably leads to hematoma and possible abscess of the vocal cord with corresponding morbidity for the patient.

The basic problem with laryngeal surface electrodes is that the aperture created by the human glottis is triangular and the endotracheal tube is round. This creates a fundamental mismatch between the surfaces. Ideally a surface electrode should be conformational to the surface being monitored. Rotation of the endotracheal tube around its long axis creates opportunity for mismatch depending on the electrode system being used.

An additional problem is that the length of the laryngeal electrode must be positioned against the edges of the vocal cord that are essentially in a plane at right angles to the endotracheal tube. Depending on the length of the electrode and the size of the patient's trachea and larynx, there is a natural tendency to displace the electrode too far into the trachea, essentially missing the vocal cords altogether. Once the endotracheal tube is in the patient and the patient is positioned for surgery and drapes applied, it is very difficult to visualize the endotracheal tube, electrode and vocal cords to verify positioning. In addition, with the gradual warming of the endotracheal tube in the patient's body, the tube becomes softer and more flexible and tends to reposition, perhaps disadvantageously for the position of the electrode. Unless a verifiable electrophysiological test of electrode performance could be designed (essentially some form of evoked test that would allow the surgeon to ping the system) this will have to be visual. The best solution and one of the objects of this invention is a continuous visualization of the electrode/larynx surface junction with a fiberoptic video system. A small fiberoptic video cameras is ideal for this purpose. In the device of this invention, the camera would be fixed at the back of the endotracheal positioning device with its field of view showing the top of the device and the larynx above it. This configuration would be assistive in initial intubation and placement of the electrode and would provide additional safety against rotation of the electrode and possible pressure points being placed on the vocal cords.

There has been no easy method for positioning surface electrodes against the muscles of the vocal cords. The present practice is more or less positioning in a trial by error method. The electrodes are moved about until they are properly positioned. Even after they were positioned, any movement by the patient or movement of the endotracheal tube could and most often would reposition the electrodes. The movement results in repositioning the electrodes and adding significant delays in the procedure.

An object of the present invention is to provide a positioning device for precisely and easily positioning surface electrodes for laryngeal monitoring. The placement of surface electrodes against the vocal cords in the glottic chink with the positioning device of this invention is a simple procedure. The learning curve is greatly reduced, and the ease and accuracy of placement is greatly enhanced.

Still another object of the present invention is to provide a device that can be adapted to use either permanent attached surface electrode(s) or a disposable surface electrode(s).

Yet another object of this invention is to provide an additional benefit of combining continuous EMG monitoring of the larynx with continuous visualization of the vocal cords during the procedure using fiber optic laryngoscope.

To accomplish the foregoing and other objects of this invention there is provided an endotracheal positioning device described below and illustrated on the accompanying drawings.

### SUMMARY OF THE INVENTION

This invention is an endotracheal positioning device that inserts into the glottic chink (area between the human true vocal cords) and provides a platform for a laryngeal surface electrode(s) and for receiving a flexible fiber optic laryngoscope. The endotracheal positioning device generally conforms to the triangular anatomic space of the glottic chink.

The endotracheal positioning device is an elongated triangular shape device having a superior surface that projects towards the junction of the true vocal cords (or anterior commissure of the larynx) and a posterior surface that projects towards the endotracheal tube or the posterior-interior surface of the cricoid cartilage (or posterior commissure of the larynx), and two lateral surfaces that project anterior lateral on either side toward the vocal cords and provide a line of approximation that is in conformity to the anatomy in the intubated state. The device has generally straight right and left lateral surfaces that extend from the superior projection to the lateral projections bilateral and that conform to the line of the leading edge of the vocal cord on each side.

The lateral surface of the device has a surface of appropriate dimensions for attachment of conductive plates known as laryngeal surface EMG electrodes that sense the electrical signals coming form the vocal cords and the other laryngeal muscles and transmit these signals by wires to an electronic unit known as an electromyographic recorder. The endotracheal positioning device has a center opening or channel that may wrap around, slide over, or may otherwise be attached to an endotracheal tube or to a high pressure ventilatory apparatus of any type. The endotracheal positioning device may be positioned either by movement of the endotracheal positioning device in conjunction with or separately from the endotracheal tube or high pressure ventilatory apparatus. Movement and positioning is accomplished by sliding on the endotracheal tube or to a high pressure ventilatory structure in the axial line, and/or about the axis by insertion and rotation on the tube so as to most favorably conform to and rest the lateral surfaces against the larynx.

The endotracheal positioning device has an attachment or docking recess on its proximal (toward the mouth) aspect for an actuating rod or a fiber optic laryngoscope, a separate medical device that encompasses a flexible light cable for both illuminating and viewing. The junction between the device and rod or cable is constructed so as to allow the rod or cable to be used manually for positioning the device relative to the endotracheal tube and with respect to the larynx, and that allows the continuous viewing of the larynx for positioning, insertion, or continuous monitoring of the vocal cords during surgical procedures. Viewing and monitoring is done continuously with the endotracheal positioning device of this invention.

The above mentioned and other objects, and features of the present invention will be better understood and appreciated from the following detailed description of the main embodiment thereof, selected for purposes of illustration and shown in the accompanying drawings.

### BRIEF DESCRIPTION OF THE DRAWINGS

FIG. 1 shows the endotracheal positioning device in cross section with an endotracheal tube positioned within the glottic chink between the vocal cords.

FIG. 2 shows a side view of the endotracheal positioning device with a surface laryngeal electrode attached.

FIG. 3 shows another side view of the endotracheal positioning device.

FIG. 3A-3C shows cross-sectional views across the endotracheal device of FIG. 3.

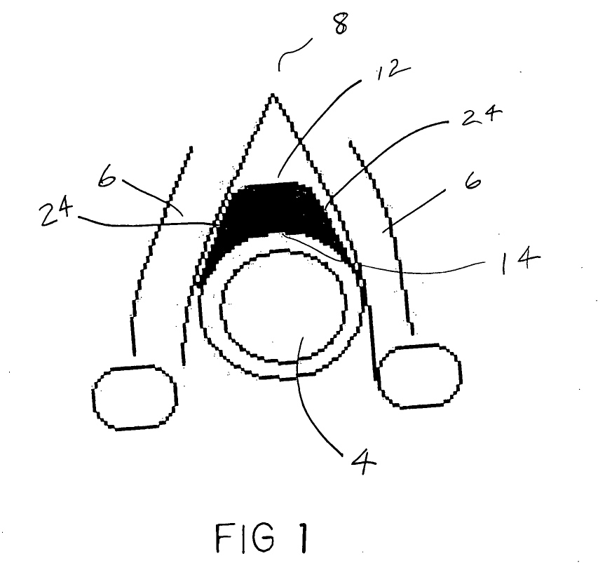

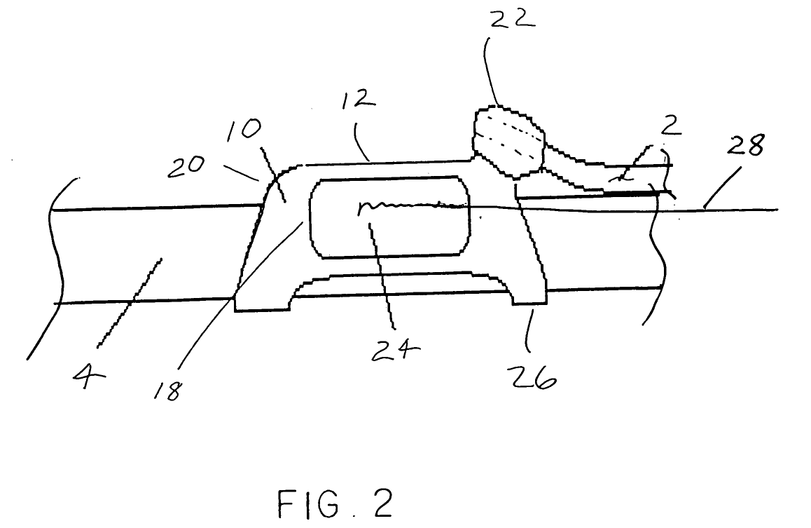

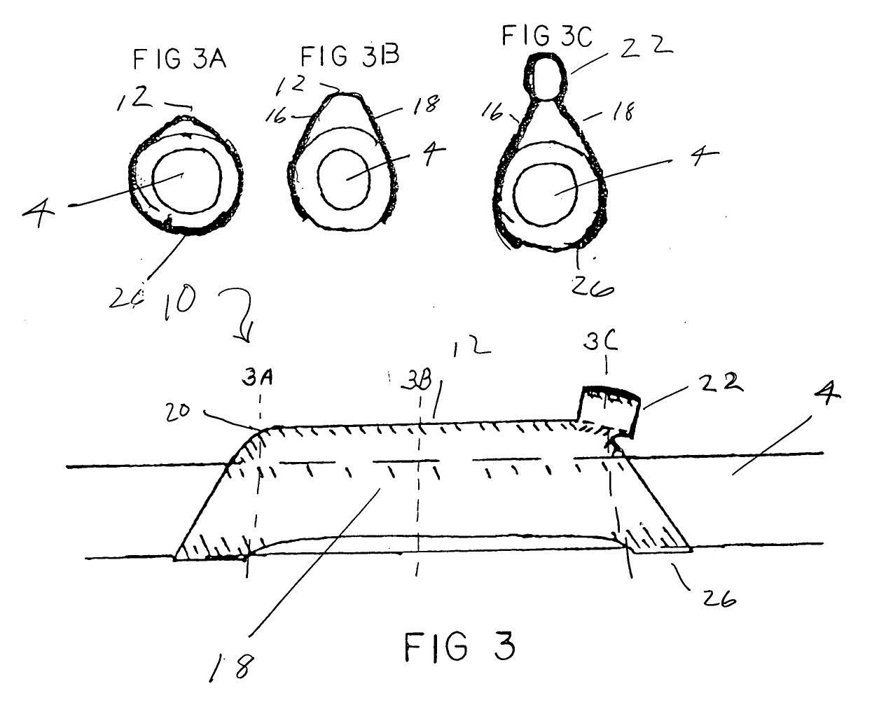

### DETAILED DESCRIPTION

Referring now to the drawings, there is shown the preferred embodiment of the endotracheal positioning device 10 of this invention for carrying and positioning laryngeal surface electrode(s) 24 and for receiving a fiber optic video device 2 for continuous monitoring of the larynx. The preferred embodiment and the best mode contemplated of the endotracheal positioning device 10 of the present invention are herein described. However, it should be understood that the best mode for carrying out the invention hereinafter described is offered by way of illustration and not by the way of limitation. It is intended that the scope of the invention include all modifications that incorporate its principal design features.

The endotracheal positioning device 10 of this invention is inserted into the glottic chink between the human true vocal cords 6. It provides a platform for a laryngeal surface electrode(s) 24 and for a flexible fiber optic laryngoscope 2. The endotracheal positioning device 10 generally conforms to the roughly triangular anatomic space of the glottic chink.

The endotracheal positioning device 10 is an elongated triangular shape device, approximately 60-65 millimeters (mm) in length in the preferred embodiment. However, the length could be greater or lesser depending on the specific requirements. The superior proximal 20 and superior distal 22 aspects are rounded so as to facilitate insertion and removal without injuring the subject human patient. The superior surface is somewhat shorter than the posterior surface.

A superior surface 12 at the apex of the triangular shape is somewhat convex and approximately 5 mm across with a length of approximately 45 mm. The superior surface 12 projects towards the junction of the true vocal cords (or anterior commissure 8 of the larynx).

A posterior surface 14 projects towards an endotracheal tube 14 or the posterior-interior surface of the cricoid cartilage (or posterior commissure of the larynx) and has a length of 60-65 mm in the preferred embodiment. The inferior surface may be shortened so as to partially encompass the endotracheal tube or may continue as to completely wrap around the endotracheal tube 4.

Two lateral surfaces 16 and 18 project anterior lateral on either side (right and left) toward the vocal cords 6 and provide a line of approximation that is in conformity to the anatomy in the intubated state of the larynx. The two lateral surfaces are generally straight right 16 and left 18 lateral surfaces that extend from the superior surface 12 to the posterior surface 14 primarily bilateral and conform to the line of the leading edge of the vocal cord 6 on each side. The lateral surface(s) 16 and 18 of the endotracheal positioning device 10 has a surface of appropriate dimensions for receiving surface laryngeal electrodes 24. In the preferred embodiment the dimensions are approximately 8-12 mm in height and 40-50 mm in length. Again the exact dimension are determined by the size requirements and the size of the patient. The surface laryngeal electrodes 24 sense the electrical signals coming form the vocal cords 6 and the other laryngeal muscles and transmit these signals by wire(s) 28 to an electronic unit known as an electromyographic recorder. A endotracheal positioning device 10 lateral surfaces 16 and 18 will accommodate either a permanently attached or a disposable version of the laryngeal surface electrode(s) 24 for purpose apposing the vocal cords 6 for laryngeal electromyographic monitoring.

The endotracheal positioning device 10 has a center axial channel(s) 26, recesses, or opening(s)(hereinafter referred to as a channel for ease of discussion) at the posterior surface 14. The channel can be of various diameters and conformations to accommodate an endotracheal tube 4 or other positive pressure ventilation apparatus of various diameters and conformations. The channel 26 may wrap around, slide over, or may otherwise be attached to an endotracheal tube 4 or to a high pressure ventilatory apparatus of any type.

The channel 26 is defined by a concave surface through the axial length (distal to proximal) on the posterior surface 14 and the posterior facing edges of the lateral surfaces 16 and 18. At the proximal and distal ends the channel may continue around to form a ring. The ring shape is preferred in that it easily slides over the endotracheal tube without twisting and rotating. The channel 26 is conformed so as to either adhere to the endotracheal tube or to facilitate sliding of the device in the axial dimension and rotation of the device around the endotracheal tube, such axis for precise positioning of the device with respect to the larynx. The channel 26 may also be closed by tension, clasps, or by overlying the opening with an attachment laryngeal surface electrode or other adhesive band.

The endotracheal positioning device 10 may be positioned either by movement of the endotracheal positioning device in conjunction with or separately from the endotracheal tube 4 or high pressure ventilatory apparatus. Movement and positioning is accomplished by sliding on the endotracheal tube or on a high pressure ventilatory structure in the axial line, or about the axis by insertion and rotation on the tube so as to most favorably conform to and rest the lateral surfaces against the larynx. The endotracheal positioning device 10 mates the laryngeal surface electrode to the endotracheal tube and provides possible mechanical positioning options to the clinician and possible auto positioning by providing free movement with respect to the endotracheal tube

The endotracheal positioning device 10 has an attachment or docking recess 22 on its proximal (toward the mouth) aspect for an actuating rod or a fiber optic laryngoscope 2 (fiberoptic video device). The docking recess 22 has an aperture of approximately 3 mm in diameter for receiving the end of the fiber optic cable. The fiber optic laryngoscope 2 is a separate medical device that encompasses a flexible light cable for both illuminating and viewing. The docking recess 22 may have an additional locking aspect that is keyed to the actuator rod or fiber optic cable to prevent inadvertent removal. The junction between the device and rod or cable is constructed so as to allow the rod or cable to be used manually for positioning the device relative to the endotracheal tube and with respect to the larynx, and that allows the continuous viewing of the larynx for positioning, insertion, or continuous monitoring of the vocal cords during surgical procedures. The optical attachment or docking device can be used to assist in positioning, verifying of positioning, and for direct observation of the vocal cords during the surgery as an ancillary form of monitoring. The endotracheal positioning device 10 may be used with or without a fiber optic device 2 attached.

In Operation: The anesthesiologist would apply the endotracheal positioning device to the endotracheal tube selected. The attachment laryngeal surface electrode would be applied to the endotracheal positioning device. The fiberoptic video camera would be secondarily applied to the back of the endotracheal positioning device. The patient is intubated with the endotracheal tube and the video camera is used to position the laryngeal surface electrode perfectly between, and in apposition to the vocal cords. The EMG unit is attached to the laryngeal surface electrode lead wires and the case is begun. During the operation, the EMG signal is displayed on a video screen below the video image of the larynx, both being capable of continuous monitoring by the surgeon and anesthesiologist. As the surgeon approaches the nerve and begins to get warning tones, the anesthesiologist alerts to the video display and assist the surgeon in final verification of the nerve location. At the end of the case the surgeon is able to perform integrity testing to verify the nerve's continued functioning. The anesthesiologist is able to use the video image of the larynx and the free running EMG signal from the larynx to help with depth of anesthesia analysis during the case and at the end of the procedure.

In summary: The endotracheal positioning device with laryngeal surface electrode(s) optimizes the positioning of the electrode(s) to the vocal cords and an indwelling fiberoptic video camera provides for verification of the positioning of the electrode(s) and allows visual monitoring of cord motion. The system will create a monitoring environment where audio warning, integrity monitoring, and visual monitoring of the laryngeal nerve function will be seamlessly employed by the surgeon to protect the patient.

Having described the invention in detail, those skilled in the art will appreciate that modifications may be made of the invention without departing from the spirit of the inventive concept herein described.

Therefore, it is not intended that the scope of the invention be limited to the specific and preferred embodiments illustrated and described. Rather, it is intended that the scope of the invention be determined by the appended claims and their equivalents.

What is claimed is:

1. A endotracheal positioning device comprising:
an elongated generally triangular shape device sized to be inserted into the glottic chink between the human vocal cords having;
a superior surface at an apex of the generally triangular shape,
a posterior surface,
right and left lateral surfaces projecting from the superior surface to the posterior surface, the lateral surfaces having appropriate dimensions for attachment of laryngeal surface electrodes, and
a center opening or channel at the posterior surface that may wrap around, slide over, or may otherwise be attached to an endotracheal tube or to a high pressure ventilatory apparatus of any type.

2. The endotracheal positioning device as set forth in claim 1 further comprising a docking recess on a proximal aspect of the device for receiving an actuating rod or a fiber optic laryngoscope.

3. An endotracheal positioning device comprising:
an elongated triangular shape device having
a superior surface,
a posterior surface,
right and left lateral surfaces projecting from the superior surface to the posterior surface, having appropriate dimensions for attachment of laryngeal surface electrodes,
a center opening or channel at the posterior surface that may wrap around, slide over, or may otherwise be attached to an endotracheal tube or to a high pressure ventilatory apparatus of any type, and
a docking recess on a proximal aspect of the device for receiving an actuating rod or a fiber optic laryngoscope.

4. An endotracheal positioning device comprising:
an elongated generally triangular shape device sized to be inserted into the glottic chink between the human vocal cords having;
a convex superior surface at an apex of the generally triangular shape for projection towards the anterior commissure of the larynx,
a posterior surface having a concave interior surface through the axial length corresponding to a shape of an endotracheal tube,
right and left lateral surfaces projecting from the superior surface to the posterior surface, the lateral surfaces for attachment of laryngeal surface electrodes for positioning the laryngeal surface electrodes in contact with the vocal cords, and
a center opening or channel at the posterior surface defined by the concave interior surface and the posterior edges of the lateral surfaces, that may wrap around, slide over, or may otherwise be attached to an endotracheal tube or to a high pressure ventilatory apparatus of any type, and
a docking recess on a proximal aspect of the device with an aperture for receiving an actuating rod or a fiber optic laryngoscope to provide continuous video observation of the larynx and vocal cords during a procedure.
